(19) United States
(12) Patent Application Publication
(10) Pub. No.: US 2008/0147150 A1
(43) Pub. Date: Jun. 19, 2008

(54) Medical laser device

(76) Inventors: Zhenhong Xiong, Beijing (CN); Shaorui Du, Beijing (CN); Ruwang Li, Beijing (CN); Guangtao Yao, Beijing (CN)

Correspondence Address: LAU & ASSOCIATES, LLC;MICHAEL N. LAU, 2121 EISENHOWER AVENUE, SUITE 503A, ALEXANDRIA, VA, 22314, US

(21) Appl. No.: 11/641,023

(22) Filed: Dec. 19, 2006

(51) Int. Cl. A61B 18/20; A61N 5/067

(52) U.S. Cl. 607/93; 604/19

(57) ABSTRACT

A medical laser device comprises the laser sources, output ports, a group of optic fibers and a laser beam spatial distribution apparatus. Wherein the device has at least two optic fibers or optic fiber cores to transmit the laser energy from the laser sources to the distal end and a spatial distribution of the laser beams are shaped with the help of apparatus of inclined optic fiber terminations and mechanical means. This medical laser device can resects unwanted material in body lumen piece by piece.

### FIELD OF THE INVENTION

The present invention relates to medical lasers or laser surgical apparatus to resects unwanted parts in body lumens piece by piece through a spatially distributed laser beams.

### BACKGROUND INFORMATION

Lasers are quite common in health-care industrial to perform minimally invasive surgical procedures such as cutting, ablation and coagulations. FIG. 9 illustrates the typical block configuration of laser medical devices consisting of reflection mirror 1′, cavity 2′, pumping lamp 3′ driven by electrical power supply 11′, laser crystal rod 4′, output mirror 5′ to set up the basic laser source (100). Among the laser beam path a beam splitter 6′, an aiming beam source 7′ perpendicular to the laser direction are placed. The laser energy are coupled through fiber coupler 8′ (200) and been transmitted into surgical fiber 9′ (300) to distal terminals 10′ (400) to perform the surgical procedures. To be simple the laser radiation or beams from the laser resonator are transmitted through fibers 300 to the distal end of laser energy delivery system (400).

Current surgical lasers are classified into two operation modes: CW (continues wave) and pulsed. Compared to CW, pulsed lasers character non-consisting radiation in time domain. The laser beams are time-shaped such as pulse width of 200 us.

For many current minimally invasive applications, surgeries are through the endoscope procedures. The flexible fibers of the surgical lasers can go through the conduit of the endoscope to reach the surgical region.

The green PVP laser system of Laserscrope features photo-selective vaporization of Prostate and uses side emission optic fibers. Dornier Lasertrode patent and ScatterFree patent of Laser Peripherals character specially designed optical parts integrated in the optic fiber distal end to vaporize the tissues. All these surgical lasers must vaporize every part of the unwanted tissues liking the painting process and thus take a lot of time. Another side effects of the above mentioned lasers is that no any life tissues for examining after the surgery.

The HoLEP (Holmium Laser Enucleation of Prostate) introduced by Lumenis as illustrated in FIG. 11 uses holmium laser to resects the prostate into its anatomic lobes and then place the lobes into bladder instead of vaporizing all the prostate adenoma. Although HoLEP features significant reduction of surgical time, some problems have been arisen. A transurethral tissue morcellator must be used to fragment the lobes helping to remove this adenomas through morcellator from the bladder, which causes safety problems. Also HoLEP is very hard to mast and posses high learning curve. The other problem for HoLEP is that usually 80 w laser power is required to perform the surgery, which approaches the maximum power the current fiber 300 allowed for transmission.

People world wide are trying to improve the surgical laser devices and hence to improve the surgical performance. However all the efforts are limited to single fiber or more specifically speaking no any efforts have been applied for spatial arrangement of the laser beams to have the effects similar to electrical resectoscope. In other words, in electrical resectoscope procedures, the tissues are resected piece by piece and thus less surgical time and testing samples can be obtained.

In resectoscopes, the electrodes have developed from single pole to circular pole as shown in FIG. 10.

### SUMMARY OF THE INVENTION

An objective of present invention is to configure the laser beams spatially which allow direct resection of unwanted materials in body lumen piece by piece in order to save operation time and take samples of unwanted parts of body lumen for examining.

The surgical device of the invention consist of lasers 100, laser output ports 200, optic fibers 300 and laser beam spatial distribution apparatus 400. In order to realize the spatial distribution of the laser beams, multiple optical fibers 300, multiple laser output ports 200 in accordance with the number of the optical fibers, optic fiber distal terminations 400 and some mechanical means are equipped. The multiple optical fibers 300 are plugged into endoscopic conduit simultaneously.

An embodiment of the devices can only has one laser source 100 but equipped with at least two laser output ports 200.

An embodiment of the devices may have two or more laser sources 100, these laser sources 100 can be identical or non-identical in terms of output characters or physical parameters.

An embodiment of the device has only one complex optical fiber with at least twin or more cores 310.

An embodiment of the device has optical fibers either straight or side emission or both of them.

An embodiment of the device has two side-emission optical fibers 300+400 to form a V-type surgical laser scalpel like circular electrodes as shown in FIG. 10.

An embodiment of the device has three side-emission optical fibers to form a funnel-shape surgical laser scalpel. The laser beam spatial distribution apparatus of an embodiment of the device have been constructed in term of easy to use, performing surgical procedure rightly.

The advantages of the present invention include:

1. Good Resection: Very good resection, be safe and efficient;

2. Good Efficiency: same time more laser beams and energy transmitted, more working areas happened at the same time;

3. Flexibility: more laser sources 100 and optical fibers 300 used, providing flexibility for different power, different resection patterns.

4. Examining sample: the life sample of the unwanted materials can be obtained.

### BRIEF DESCRIPTION OF THE DRAWINGS

FIG. 1 The schematic diagram of present invention with multiple laser sources 100, multiple optical fibers 300, multiple output ports with quantity in accordance with optical fiber numbers and laser beam spatial distribution apparatus 400.

FIG. 2A is a schematic view of an embodiment with two cores 310 in one optical fiber 300 of the invention.

FIG. 2B is the radial cross-section view of the optical fiber 300 taken in line 1-1.

FIG. 3A is a schematic diagram of an embodiment with two optical fibers 300 of the invention.

FIG. 3B is a schematic diagram of an embodiment with three optical fibers 300 of the invention.

FIG. 3C is a schematic diagram of an embodiment with two laser sources 100 and two optical fibers 300 of the invention.

FIG. 4A is the longitudinal view of the optical fiber 300 with fiber coating 320 and fiber core and cladding 310.

FIG. 4B is radial cross-section view of the optical fiber 300 taken in line 1-1.

FIG. 4C is the longitudinal view of the inclined terminal of optical fibers 300 with line 2-2 the normal of the end face.

FIG. 5A is the longitudinal view of the inclined terminal of optical fiber 300 with focus lens 405 and line 2-2 the normal of the end face. Optic lens 405 will focus the laser beams from the inclined terminal in the body lumen.

FIG. 5B is a schematic longitudinal cross-section view of angled optical fiber tip in accordance with the present invention. The coating 402 is the total reflective mirror for the laser beams. Focus lens 405 will focus the laser beams reflected from coating 402 in the body lumen.

FIG. 5C is a side view of the laser probe which reflects the laser beam in the terminal of the optical fiber invented by Laser Peripherals.

FIG. 6 is a perspective view of an embodiment of laser beam spatial distribution of a surgical device of the invention with simple integration of two straight terminal face fibers 300.

FIG. 7A is a schematic view of an embodiment of laser beam spatial distribution apparatus of a surgical device of the invention with two optical fibers configured to fire V-shape laser beams. Apparatus 301 is an embodiment of the mechanical parts with direction and position mark 302.

FIG. 7B is cross-section view of the embodiment of laser beam spatial distribution apparatus of the surgical laser device of the invention taken in line 1-1. The amplified view of fiber terminals with focus lens is also given in the left side of the figure.

FIG. 7C is cross-section view of the embodiment of laser beam spatial distribution apparatus of the surgical laser device of the invention taken in line 1-1. The amplified view of fiber terminals without focus lens is also given in the left side of the figure.

FIG. 8 is a perspective view of an embodiment of laser beam spatial distribution apparatus of a surgical device of the invention with three optical fibers 300 configured to fire core-shape laser beams. Part 303 is a mechanical apparatus with some direction and position marks on it.

FIG. 9 is a schematic diagram of a typical medical laser system.

FIG. 10 shows the popular circular electrode of resectoscopes of medical products.

FIG. 11 is a perspective view of the enucleation of the prostate by Luemenis HoLEP.

The various aspects of present invention will be described in detail with the help of the illustrating figures in following.

### DETAILED DESCRIPTION OF PRESENT INVENTION

As illustrated in FIG. 1, one embodiment of the medical device of present invention consists of laser sources 100, laser beam output ports 200, optical fibers 300 transmitting laser energy and laser beam spatial distributing apparatus 400. The key issue of present invention is the medical device has at least one laser source, a group of optical fibers with optically integrated terminals intended to form spatial distribution of the laser beams and plugged into the endoscopic conduit simultaneously. The laser output port quantities are in accordance with the number of the optical fibers.

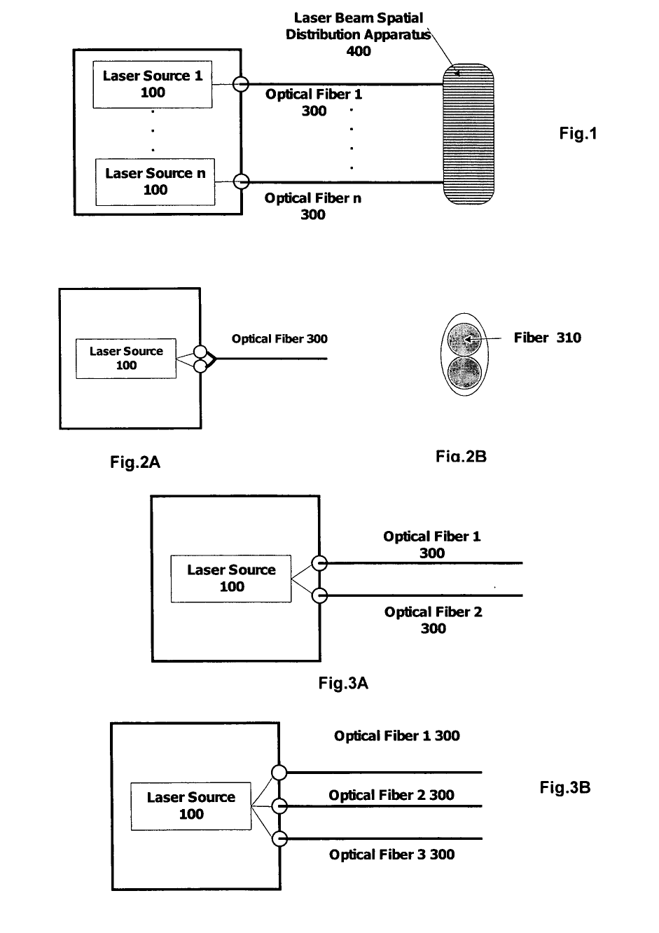

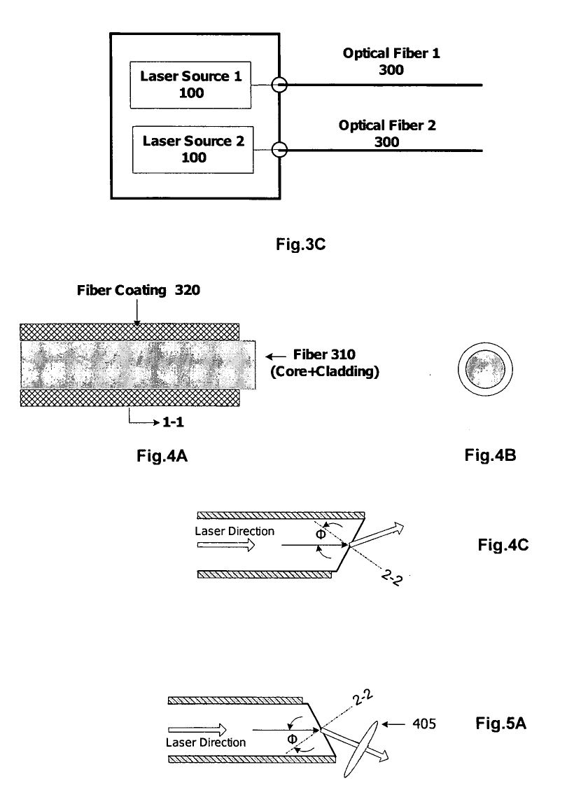

As illustrated in FIG. 1 the embodiment of the surgical laser system may has various quantities in laser sources 100 and optical fibers 300 which transit the laser source energy. In addition to FIG. 1, FIG. 2,3 show the examples of the embodiments of the device of the invention. The device of the invention in FIG. 2A has only one laser source, one complex optic fiber but two fiber cores 310 and two laser output ports 200, which means one laser source but multiple output ports and optical fiber cores. The device of the invention shown in FIG. 3A has only one laser source 100 but two output ports 200 and two optical fibers 300. While in FIG. 3B there are one laser source 100 but three output ports 200 and three optical fibers 300 in one embodiment of the device of the invention. FIG. 3C illustrates the device has two laser sources 100 and two optical fibers 300.

There are various configurations of the laser source 100 for the embodiment of the device of the invention. Take an example of two laser sources 100 in combination, these two laser sources 100 may be either identical or non-identical in output and parameters; Two laser sources may operate either in the mode of CW or in pulse mode. Or one laser source 100 operates in CW while the other in pulse. When two laser sources 100 are pulse laser, they can be either same or different in frequency, pulse width, phase etc. While the phase between each other can be related and adjusted. The same reason the laser source quantity of the device of the present invention can be as many as three or more.

The device of the present invention may have many different combinations of optical fibers 300 in terms of construction or configuration. A group of optical fibers 300 should at least have two single fibers 310, or have only one complex fiber but with at least two cores 310 or above. A group of fibers 300 can be the combinations of either the single fiber 300 and complex fiber 300 or just the different complex fibers 300. The optical fibers of the device of the invention can be either the same or different in core diameters, materials, numeric aperture. Their optical fiber terminals can be either the same or different in terms of the inclined face or straight face.

For side emission optical fiber 300 of the laser beam spatial distribution apparatus of the invention, there are two embodiments of the fiber tip face of the device. One is to use the principle of refraction of light as shown in FIG. 4C. Considering the environmental of the endoscope and the optical fiber tip of the surgical laser is surrounded with water, we can define the maximum angle which is between the optical axis of the optic fiber and the normal 2-2 of inclinded terminal by following equation.

Φₒ=arc sin(n_{w}/n_{f})=62.46°

Where n_{w} is refraction rate of the water and n_{f} is refraction rate of quartz, Φₒ is the maximum angle of the inclined face. So the angled face must be defined as 0<Φ≦Φₒ.

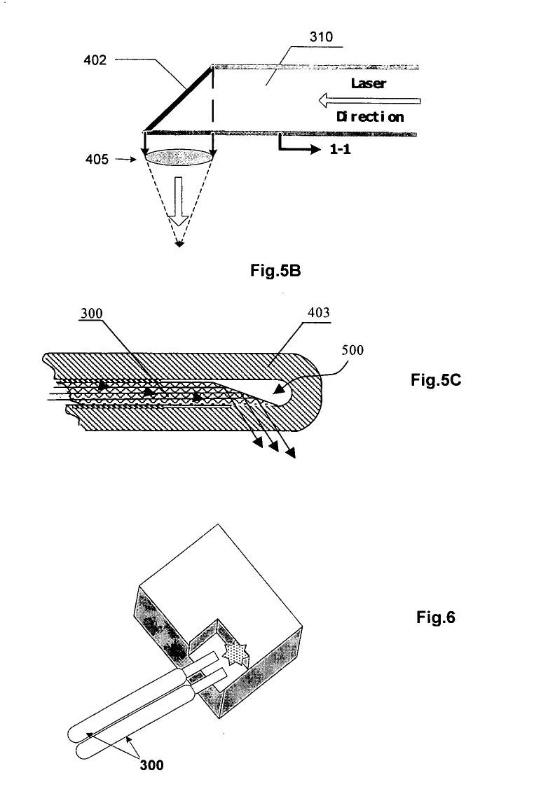

Another common practice of the optic fiber end face is to apply a total-reflection coating into the end face of the optical fiber illustrated in FIG. 5B. The patent products ScatterFree of Laser Peripherals is an example of side-emission optical fibers.

The distal fiber terminals can be straight or inclined or combination of both in the laser beam spatial distribution apparatus of the invention.

There are some limitations on the quantity of the fibers and distance between the fibers. If the inner diameter of the endoscope is D, the distance between fibers is L, the L is defined by 0≦L≦D−d.

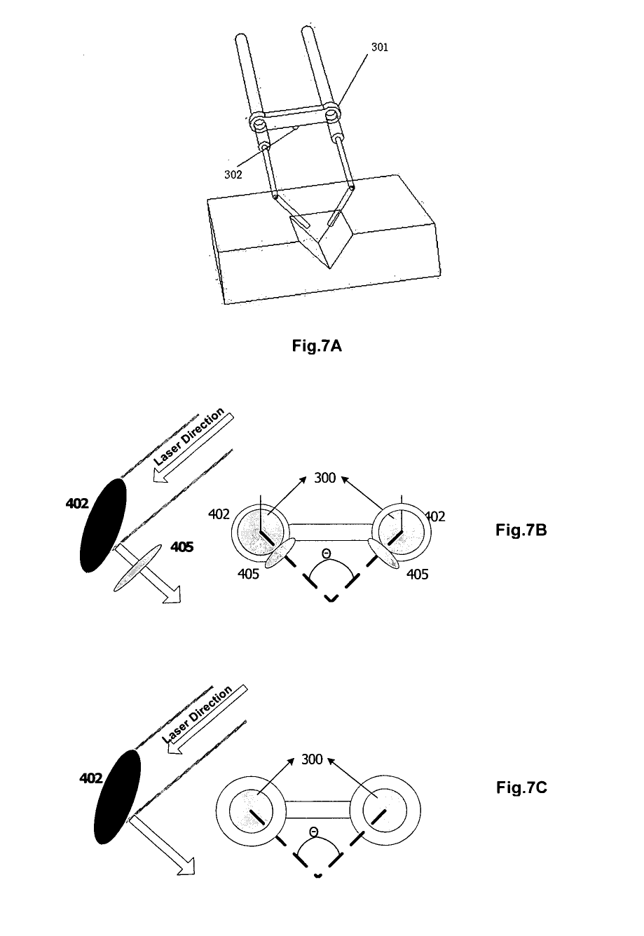

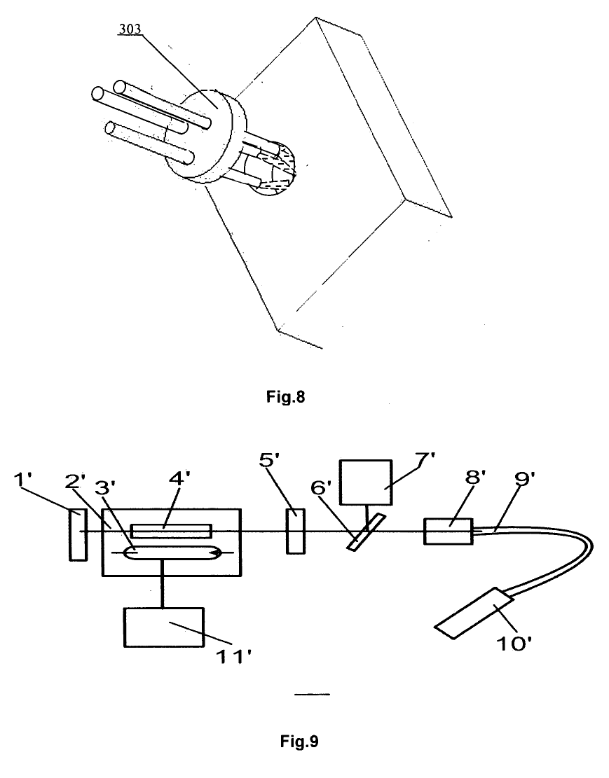

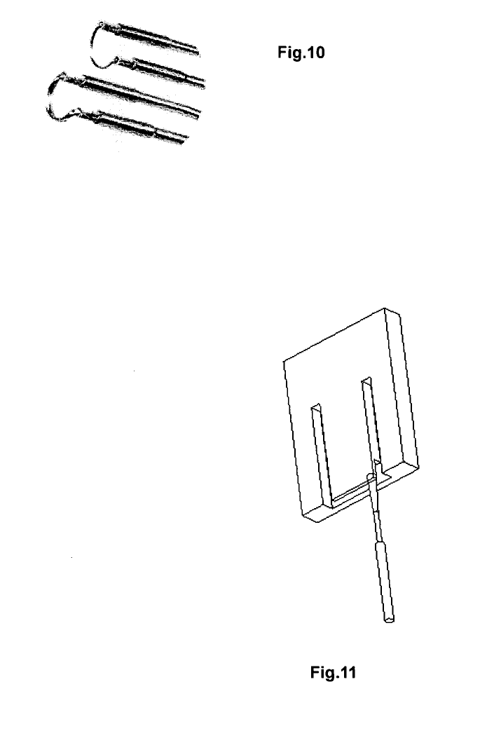

The three embodiments of laser beam spatial distribution apparatus of the invented device are shown in FIG. 6, FIG. 7A, 7B, 7C and FIG. 8. One of them is of two single fibers to transmit the laser energy with straight distal terminations as shown in FIG. 6. This apparatus can be used in applications requiring higher laser power. The second one is of two single optical fibers with specially designed terminal probes to form a V-shaped laser beams as shown in FIG. 7A. Some necessary mechanical means and markings should be used as indicated in 301 and direction as well as position mark 302. FIG. 7B shows the cross-section view of the laser beam spatial distribution apparatus with focus lens 405 and reflection coating 402 with an amplified view on the fiber terminals. The two fibers separate each other with distance L and two beams form an angle of θ. The angle θ can be changed within 0°˜180°. There is an embodiment of the laser beam spatial distribution apparatus which has no focus lens as shown in FIG. 7C. This laser beam delivery system of the invention can resects unwanted materials in a body lumen such as BPH adenoma piece by piece through movement of backward and up. The third one is of three single optical fibers with specially designed terminal probes to form a corn-shaped laser beams as shown in FIG. 8. Some mechanical means indicated as 303 should be used to configure the three optical fibers 300. This laser beam delivery system of the invention can resects unwanted materials in a body lumen such as BPH adenoma piece by piece through circular movement at certain angles.

1. A surgical laser device comprising: laser sources, laser output ports, multiple optical fibers who transmit laser energy from laser sources and an apparatus of laser beam spatial distribution. This device characters at least one laser source, a group of optical fibers that are plug into the conduit of the endoscope simultaneously and laser output ports with numbers corresponding to the number of the optic fibers. The laser beam spatial distribution apparatus has necessary mechanical means to configure the optical fibers and specially designed optical fiber distal terminals to form some pattern of laser beams.

2. The medical laser device of claim 1, wherein a group of optical fibers has at least one complex fiber with two cores.

3. The medical laser device of claim 1, wherein a group of optical fibers has at least two single optic fibers.

4. The medical laser device of claim 3, wherein a group of optic fibers comprising single core, multiple cores or combination of both single core and multiple cores.

5. The medical laser device of claim 3, wherein a group of optic fibers are completely the same, or different or partly the same and partly different in terms of characters, parameters and constructions.

6. The medical laser device of claim 1, wherein a group of fibers are either straight end face or inclined end face in distal terminations.

7. The group of optic fibers of claim 6, wherein the inclined optic fiber terminals has an angle Φ≦62.46° between the optic axis of the optical fiber and the normal of the end face.

8. The group of optic fibers of claim 6, wherein inclined end faces have the same angles or different angles or both in terms of the inclined angles.

9. The medical laser device of claim 1, wherein a laser source has at least two laser output ports to couple the laser beams into the optic fibers.

10. The medical laser device of claim 1, wherein the laser source has two or more ones.

11. The laser sources of claim 10, wherein the laser sources are either the same or different in terms of output characteristics and parameters.

12. The laser sources of claim 10, wherein the laser sources are operated in CW mode.

13. The laser sources of claim 10, wherein the laser sources are operated in pulsed mode.

14. The laser source of claim 13, wherein the laser sources are either the same or different in terms of frequency, pulse width. While the phase between the each other are related or not.

15. The laser sources of claim 10, wherein the laser sources are the combination of CW and pulsed ones.

16. The medical laser device of claim 1, wherein the laser beam spatial distribution apparatus has specially designed optical fiber terminations.

17. The medical laser device of claim 16, wherein the inclined optic fiber terminations integrated with optic lens to focus the laser beam.

18. The laser beam spatial distribution apparatus of claim 16, wherein the inclined optic fiber terminations have no optic lens integrated.

19. The laser beam spatial distribution apparatus of claim 16, wherein the laser beams are spatial shaped as V-type by two inclined fiber terminations.

20. The laser beam spatial distribution apparatus of claim 16, wherein the laser beams are spatial shaped as core-type by three or over inclined fiber terminations.

21. The laser beam spatial distribution apparatus of claim 16, wherein the optical fibers have some marking for directions and positions.

22. The laser beam spatial distribution apparatus of claim 16, wherein the necessary mechanical means configure the optic fibers and optic fiber distal terminations.

23. The laser beam spatial distribution apparatus of claim 16, wherein the necessary mechanical means have marking to identify the directions and positions.

24. A surgical laser device comprising:
a plurality of laser sources;
a plurality of laser output ports;
a plurality of optical fibers transmitting laser energy from the plurality of laser sources; and
an laser-beam spatial distribution apparatus;
wherein at least one of the plurality of laser sources, a group of the plurality of optical fibers that are plugged into a conduit of an endoscope simultaneously and a plurality of laser output ports corresponding to the plurality of optic fibers; and
wherein the laser beam spatial distribution apparatus include mechanical means to configure the plurality of optical fibers and a plurality of specially designed optical fiber distal terminals to form multiple laser beam patterns.
